(19) United States
(12) Patent Application Publication
(10) Pub. No.: US 2005/0245990 A1
(43) Pub. Date: Nov. 3, 2005

(54) Hearing implant with MEMS inertial sensor and method of use

(76) Inventor: Joseph Roberson, East Palo Alto, CA (US)

Correspondence Address: Joseph Roberson, Suite 101, 1900 University Ave., East Palo Alto, CA, 94303, US

(21) Appl. No.: 10/833,424

(22) Filed: Apr. 28, 2004

(51) Int. Cl. A61N 1/18

(52) U.S. Cl. 607/57

(57) ABSTRACT

An implant device for treating hearing disorders. In one exemplary embodiment, an implant body is dimensioned for attachment to the ossicular chain of a patient. The implant body carries a micro-encapsulated MEMS inertial sensing device that is electrically coupled by a micro-cable to an implantable signal processing system. The MEMS inertial sensor is capable of directly sensing acoustic waves transmitted through the ossicular chain. Signals from the inertial sensor are sent to the signal processing system for filtering, conditioning and amplification to thereafter be carried to a plurality of electrodes carried by a cochlear implant.

### CROSS-REFERENCE TO RELATED APPLICATIONS

This application claims benefit of the following Provisional U.S. Patent Applications: Ser. No. 60/______ filed May 1, 2003 (Docket No. JR-003) titled “Cochlear Implant with MEMS Inertial Sensor and Method of Use” and Ser. No. 60/______, filed May 1, 2003 (Docket No. S-JR-004) titled “Cochlear Implant with MEMS Piezoelectric Sensors”, both of which are incorporated herein by this reference.

### BACKGROUND OF THE INVENTION

1. Field of the Invention

The present invention relates to implantable devices for treating hearing disorders. More in particular, an exemplary embodiment of the invention comprises an implant that is surgically placed in the ossicular chain that carries a MEMs inertial sensing device for sensing and capturing vibratory displacements relating to frequencies of acoustic pressure waves, together with systems for processing, amplifying and delivering signals to the cochlea.

2. Description of the Related Art

The middle ear comprises a levered vibrating system for sound transmission from the tympanic membrane (eardrum) to the inner ear. The outer ear picks up acoustic pressure waves which are converted to mechanical vibrations by a series of small bones in the middle ear. The air-filled volume of the inner ear contains three middle ear bones or auditory ossicles: the malleus 4, the incus 6 and the stapes 8 (see FIG. 1). The malleus has a handle portion that contacts the tympanic membrane 7 and a head portion that couples with the incus. The stapes includes an arch, formed by a pair of limbs, and a footplate. The footplate communicates with the oval window that leads to the cochlea 10. As the malleus handle vibrates in response to sound waves striking the tympanic membrane, the head portion of the malleus couples the vibrations to the incus, and thereafter to the arch of the stapes. The stapes footplate in turn couples the auditory vibrations to the cochlea. The shape and structure of the ossicular chain creates a lever action within the middle ear to amplify vibrations. Thus, a greater vibrational force is generated at the oval window than at the tympanic membrane.

The inner ear consists of the cochlea 10, which has a spiral-shaped fluid-filled cavity that transforms the mechanical vibrations into vibrations in the fluid. The pressure variations in the cochlear fluid result in mechanical displacements of the flexible basilar membrane that spirals within the duct of the cochlea. The mechanical displacement of the basilar membrane provides information relating to the frequency of the acoustic signal. Hair cells are attached to the basilar membrane, which are bent according to the displacements of the basilar membrane. It is the bending of the hairs that release electrochemical substances that causes neuron firing activity at particular sites along the cochlear duct. The central nervous system transmits the signals to the brain resulting in acoustic awareness.

The hair cells in conjunction with the basilar membrane are responsible for translating mechanical information into neural information. If the hair cells are damaged, the auditory system has no way of transforming acoustic pressure waves to neural impulses, and that in turn leads to hearing impairment. The hair cells can be damaged by diseases such as meningitis, Meniere's disease and congenital disorders. Damaged hair cells can subsequently lead to degeneration of adjacent auditory neurons, and if a large number of hair cells or auditory neurons throughout the cochlea are damaged, the person with such a loss is diagnosed as profoundly deaf.

### SUMMARY OF THE INVENTION

In general, the apparatus of the present invention provides an implant that can be attached to the incus, stapes or other portion of the ossicular chain. In a preferred embodiment, the implant body carries a micro-encapsulated MEMS inertial sensor that is electrically coupled by a micro-cable to a signal processing system implanted subcutaneously behind the patient's ear. The MEMS inertial sensor directly senses acoustic waves transmitted through the ossicular chain. Signals from the inertial sensor are sent to the signal processing system for filtering, conditioning and amplification to thereafter be carried to a plurality of electrodes carried by a cochlear implant.

Of particular interest, the implant corresponding to the invention for the first time will allow for the acoustic sensor (i.e., a microphone component) to be implanted within the patient's ear. The prior art cochlear implants rely on an external microphone that is coupled to electrical leads that are surgically implanted to extend to the cochlear implant.

### BRIEF DESCRIPTION OF THE DRAWINGS

Other objects and advantages of the present invention will be understood by reference to the following detailed description of the invention when considered in combination with the accompanying Figures, in which like reference numerals are used to identify like elements throughout the disclosure.

FIG. 1 is a schematic view of the outer ear, middle ear and inner ear that show the location of the auditory ossicles and cochlea.

FIG. 2 is a schematic view of an exemplary Type “A” implant after being surgically implanted between the incus and the stapes.

FIG. 3 is a perspective cut-away view of the implant of FIG. 2 showing a MEMS piezoresistive sensor corresponding to the invention resent invention that is adapted to sense acoustic pressure waves along at least one axis.

FIG. 4 is an enlarged perspective view of the MEMS piezoresistive sensor of FIG. 4.

FIG. 5 is a more greatly enlarged perspective view of the MEMS piezoresistive sensor with its various components more clearly shown.

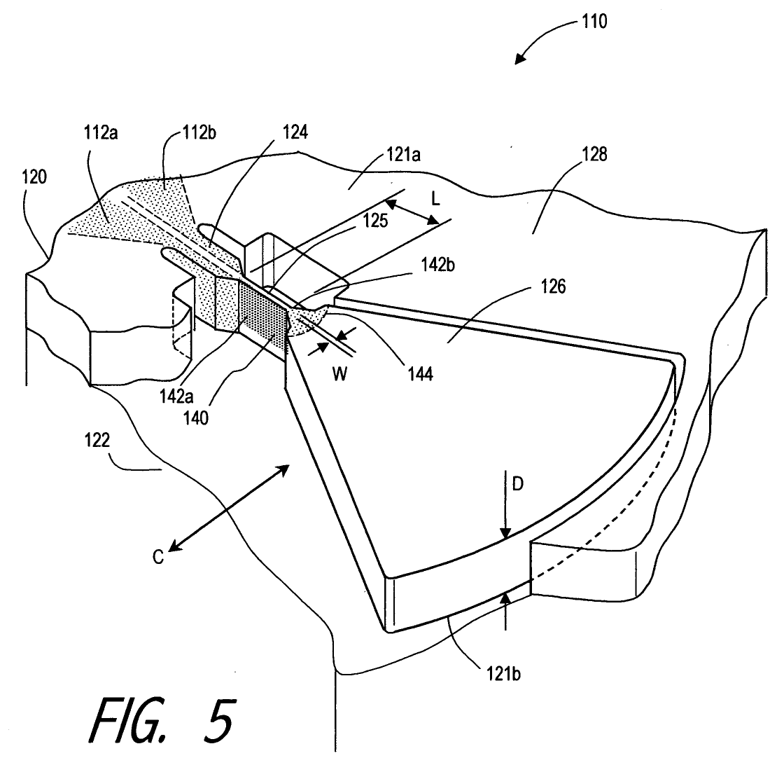

FIG. 6 is a schematic view of an exemplary Type “B” implant after being surgically attached to the incus.

FIG. 7 is a perspective cut-away view of the implant of FIG. 6 showing the MEMS piezoresistive sensor carried by the implant body.

FIG. 8 is a schematic cut-away view of a Type “C” cochlear implant of with MEMS sensors within the cochlear duct.

### DETAILED DESCRIPTION OF THE INVENTION

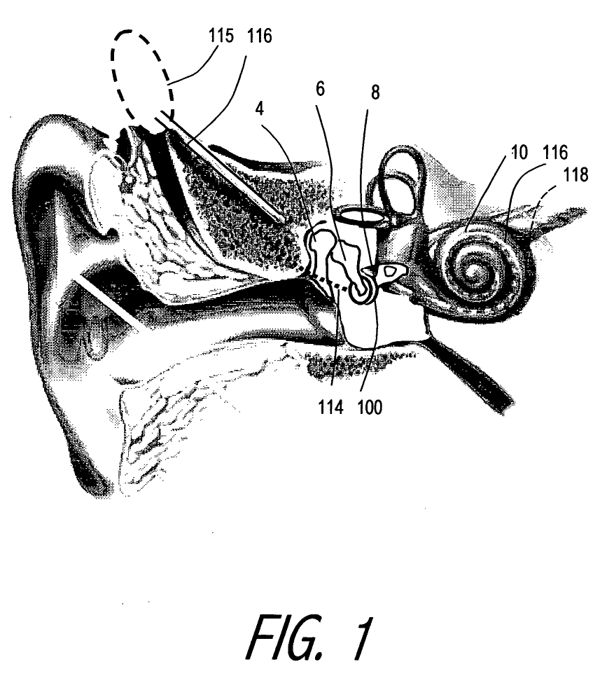

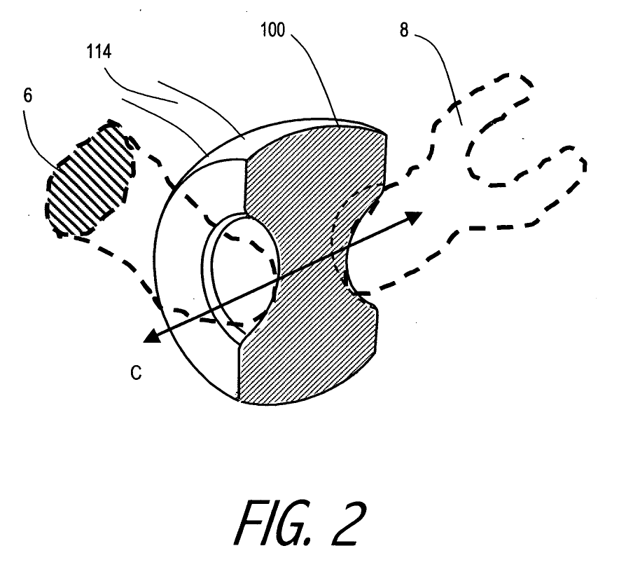

1. Type “A” implant with MEMS inertial sensor. An exemplary Type “A” implant 100 corresponding to the invention is illustrated in FIGS. 1 and 2 that is adapted for implantation between the incus and stapes. In general, the Type “A” embodiment of the invention is based on piezoresistive sensing of the displacement of a seismic mass in response to vibration in the ossicular chain. The acoustic sensor is fabricated by a MEMS process. The term MEMS (micro-electrical mechanical systems) describes the integration of mechanical elements, sensing elements and electrical elements on a common silicon substrate through microfabrication technology. While the electronics are fabricated using integrated circuit (IC) process sequences (e.g., CMOS, Bipolar, or BICMOS processes), the micromechanical components are fabricated using compatible micromachining processes that selectively etch away parts of the silicon wafer or add new structural layers to form the mechanical and electromechanical devices. The MEMS sensors and systems made by silicon micro-fabrication techniques allow a very high level of functionality, reliability, and sophistication at a relatively low cost.

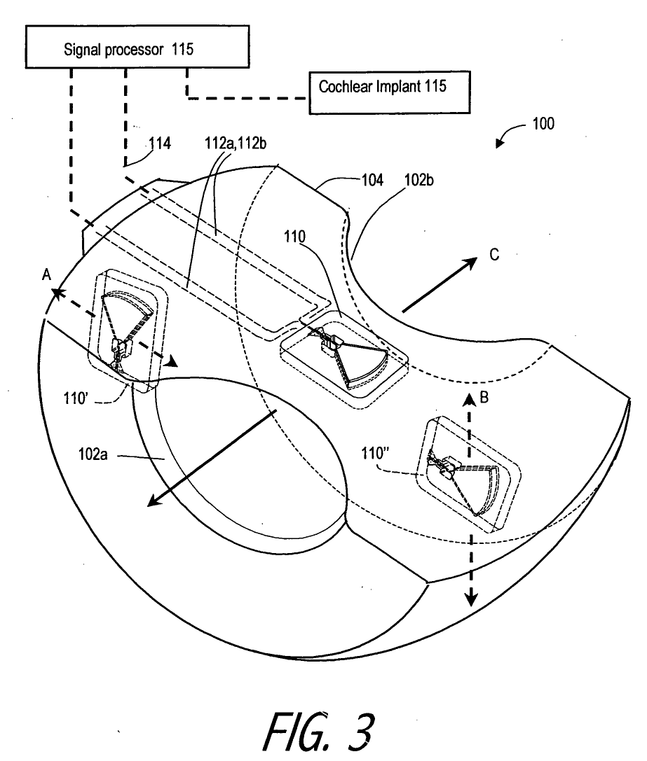

FIGS. 2 and 3 illustrate an exemplary implant 100 having a generally donut shape that defines recessed portions 102a and 102b that are dimensioned to receive the end portions of the incus and stapes. The incus and stapes can be surgically separated from one another at the I-S joint and the implant 100 is placed therebetween. The surface or body of the implant can be hydroxyapatite or any other material, alloy or polymer commonly used in ossicular implants to allow rapid fusion of the bones to the implant. As can be seen in FIG. 3, the body 104 of the implant defines three orthogonal axes indicated at A, B and C. In this embodiment, it can be understood that the primary acoustic waves will be transmitted through the ossicular chain and propagate along axis C of the implant body. At least one planar sensor 110 is carried within the implant body, in this case preferably aligned with the axis C. More preferably, the implant body carries a plurality of sensors 110, 110′ and 110″ that are adapted to sense displacements in three axes. Alternatively, a single sensor can be of a type adapted to measure acoustic waves in multiple axes.

In FIGS. 2 and 3, the sensor 110 has signal electrical leads 112a and 112b coupled thereto that in turn are carries by a micro-cable 114 to an implanted signal processing component indicated at 115 in FIG. 1. The off-chip processing component 115 is implanted under that patient's skin as is known in some existing cochlear implant system. The system 100 further has a cochlear lead 116 that is implanted to extend into the cochlear duct 118 that is similar to prior art cochlear implants. The cochlear lead 116 carries a plurality of electrodes that are adapted to stimulate the auditory nerve fibers in the cochlea at predetermined locations along the duct.

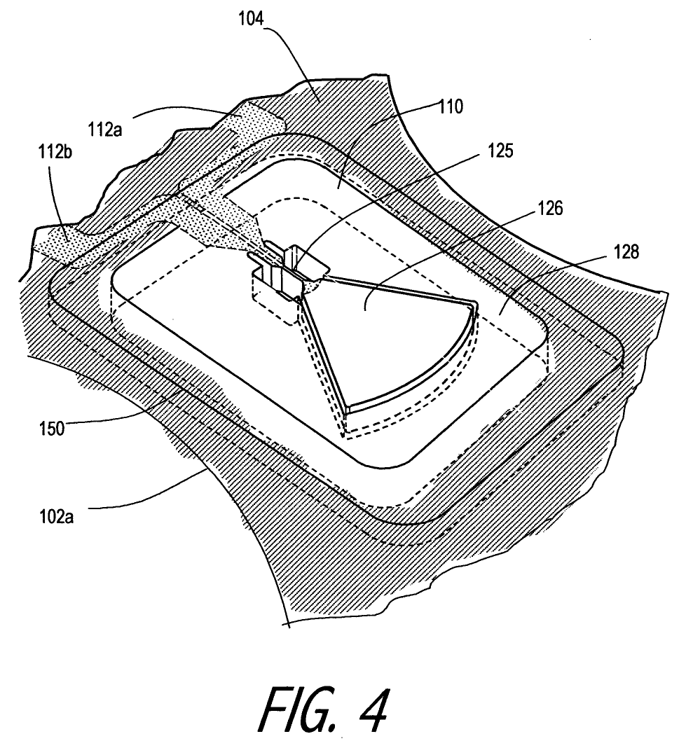

Now turning to FIGS. 3 and 4, one embodiment of MEMS acoustic sensor 110 of the invention is fabricated from a silicon wafer or body 120 with the planar device portion defining top and bottom surfaces 121a and 121b, respectively. The sensor of the invention can be fabricated generally in the dimensions and manners as described in the accelerometers of U.S. Pat. No. 6,389,899 titled “In-plane micromachined accelerometer and bridge circuit having same” to A. Partridge et al; and in A. Partridge et al., “A high performance planar piezoresistive accelerometer,” IEEE Journal of Microelectromechanical Systems, (JMEMS), vol. 9, No. 1, March 2000, pp. 58-66; and at http://micromachine.stanford.edu/˜aaronp/navAcc.html, all of which are incorporated herein by this reference. The wafer has a base layer 122 that can be any suitable thickness. The sensor etched to provide a pedestal portion 124 that extends from the base layer 120, wherein the pedestal 124 transitions into a high aspect ratio flexure or cantilever indicated at 125. A planar seismic mass 126 is carried by the flexure portion. As can be understood from FIGS. 3 and 4, the suspended planar mass 126 is undercut so that its bottom surface floats free and is deflectable relative to base 122 portion. The planar mass 126 (or proof mass) is sensitive to displacement relative to axis C. In this embodiment, the suspended planar mass 126 is wedge shaped and is caged by the surrounding silicon layer 128.

As can be seen in FIG. 4, the flexure 125 is micro-fabricated with a piezoresistive material 140 on a vertical surface 142a of the flexure. The top surface 121a of the sensor carries an electrical circuit path comprising the signal leads (doped silicon) that extend to the flexure 125. In this embodiment, the electrical lead 112a extends down the vertical surface 142a toward the base of the flexure to contact the piezoresistive material 140. The circuit then extends through the piezoresistive material 140 along one surface of the flexure and back to a conductively doped portion 144 at the top 121a of the body. The circuit then can extend along the back side 142b of the flexure 125 to couple to lead portion 112b. As can be understood from FIGS. 3 and 4, the suspended planar mass portion 126 is adapted to be displaced relative to axis C.

In FIG. 4, the dimensions of the planar sensor 110 are indicated generally. The depth D or thickness of the suspended mass 126 can range between about 10 micron and 100 microns. The flexure 125 has a width dimension W that can range between about 0.01 micron and 10 microns. More preferably, the width of the flexure 125 ranges between about 0.5 micron and 5 microns. The length L of the flexure 125 can range between about 10 micron and 10 and 100 microns. The suspended mass 126 can be any suitable shape with a wedge shape being known in the art of MEMS accelerometers. The length and width of the suspended mass 126 can range between about 10 microns and 200 microns.

The signal leads 112a and 112b and the piezoresistive doped portion 140 of the flexure form an electrical circuit in which the resistance of the circuit is varied with changes in resistance in the piezoresistor 140. In use, when the suspended mass 126 is exposed to an acceleration field, the inertia of the seismic mass will cause the sensor's silicon flexure 125 to bend and stress. The piezoresistive portion 140 at the surface of the flexure will undergo high mechanical stress and will change its value due to the piezoresistive effect in doped silicon. The detection of inertial forces is thus possible with an output signal carried via the signal leads 112a and 112b to the signal processing system 115.

The method of making the planar sensor 110 in silicon is described in U.S. Pat. No. 6,389,899 referenced above. In general, a silicon substrate is provided that carries a buried oxide layer that can be etched to provide the flexure and suspended mass floating above the base 122 (see FIG. 4). The surface of the sensor is masked and implanted with which heavily doped regions to provide the signal leads 112a and 112b. The regions 112a and 112b extend to a portion of the implant to allow connection to the signal cable 114 that extends to the signal processor 115 (see FIG. 1). The planar sensor is then masked to allow etching of the flexure 125 and suspended mass 126. The piezoresistor material 140 is implanted into the sidewalls of the flexure 125 to form a sensing system.

A suitable encapsulation technology is used to encapsulate the sensor in a package. In one embodiment, the capsule is less than about 1 mm. in its maximum exterior dimension along any axis. Preferably, the encapsulated sensor has a maximum exterior dimension along any axis of less than 0.5 mm. More preferably, the encapsulated sensor has a maximum exterior dimension along any axis of less than 0.25 mm. New wafer-scale encapsulation technologies have been developed for inertial sensors, wherein the encapsulation consists of approximately 20 micron thick cap layer 150 (see generally FIG. 3) deposited on the MEMS device during fabrication, followed by release. The resulting MEMS devices and sensors can thus be encapsulated with the complete package being less than about 250 microns along any axis.

The planar body 120 (see FIGS. 34) can carry other similar or identical piezoresistive flexures to provide matched bridge resistors and a thermal calibration resistor. In one embodiment, the sensors corresponding to the invention are made of silicon. The scope of the invention includes the use of any materials suitable for micro-fabrication processes, e.g., quartz and other crystalline materials, ceramics, and other semiconductors such as gallium arsenide.

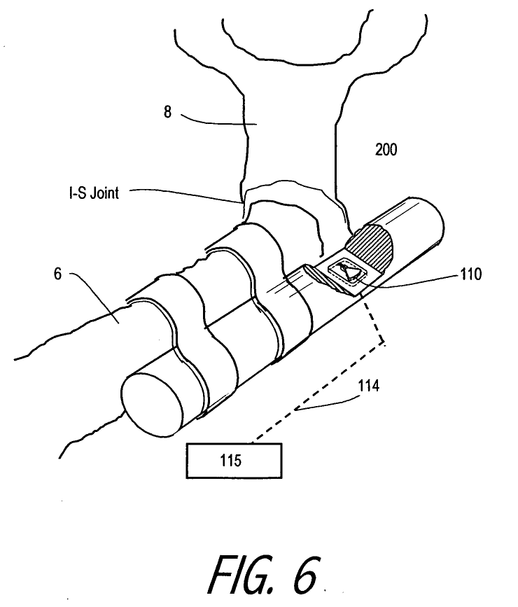

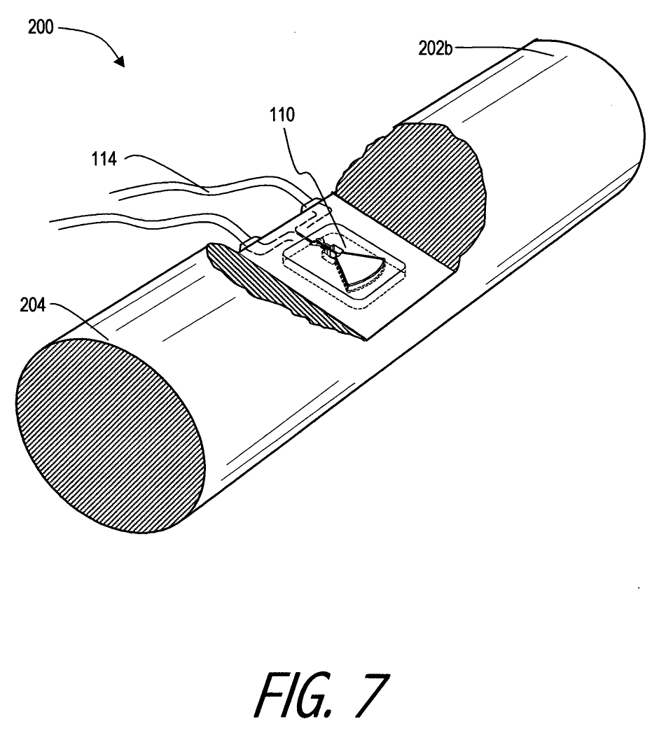

2. Type “B” cochlear implant and MEMS inertial sensor. Another embodiment of implant 200 (see FIGS. 6 and 7) can carry at least one inertial sensor similar to that of FIGS. 3 and 4, except that the cantilever or flexure 125 is doped with a piezoelectric composition. A signal lead extends from the piezoelectric composition to a signal processor as described above. In use, the displacement of the suspended mass and deflection of the cantilever will create and transmit electrical signals to the signal processor for conditioning and amplification for transmission to the cochlear implant. In other words, the sensor generates an electrical signal rather than altering the resistance within a circuit to accomplish the sensing function.

In FIGS. 6 and 7, the exemplary embodiment is attached by any suitable means such as crimps to the incus 6.

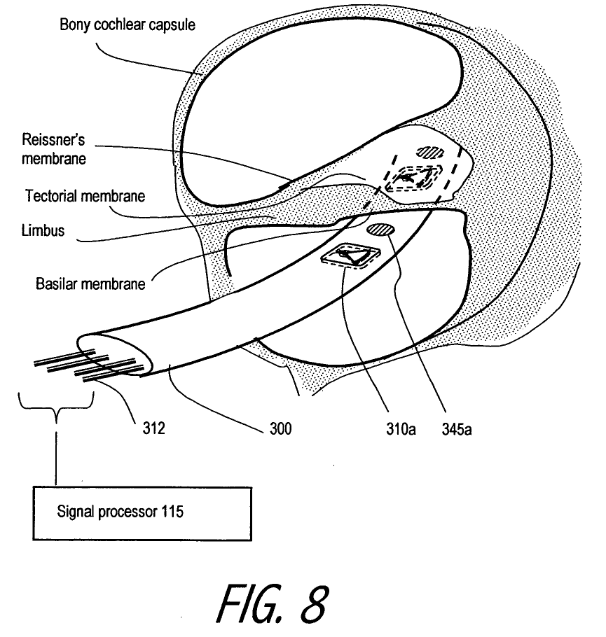

3. Type “C” implant with MEMS inertial sensors in cochlear duct member. FIG. 8 illustrates a Type “C” implant system 300 that differs from the Types “A” and “B” embodiments above. In this embodiment, the implant 300 (FIG. 8) comprises an elongated body that has a cross-sectional dimension and axial dimension for implantation in the duct of the patient's cochlea, or with portions of the implant within the duct and other portions extending within any other portion of the ear structure. Of particular interest, the implant body carries a plurality of axially spaced apart inertial sensors 310a to 310n (310 collectively) that may range in number from 1 to 25 or more, wherein each sensor 310 is adapted to detect and respond to local acoustic pressures in the cochlear duct. In a preferred embodiment, the implant body is attachable to the basilar membrane by any suitable means such as tissue adhesives, clips and the like. Thus, the implant of FIG. 8 allows the detection of acoustic pressures directly at the site of the natural transduction of pressure to neural inputs, which it is believed will allow for greatly improved detection and follow-on delivery of neural stimulation.

In the embodiment of FIG. 8, the inertial sensors are of a type similar to those of the Types “A” and “B” embodiments above. The sensors at each axial location along the duct may be singular or plural and can be oriented to respond to sound vibrations in a single axis or in 3 axes. The detection of such local pressures again results in input signals that are carried to the sound processor by circuitry 312 for conditioning and amplification to thereafter cause electrical energy delivery from electrodes 345a to 345n to the auditory nerves about the cochlear duct.

In another embodiment (not shown), the flexures of the sensors can carry a piezoelectric element to produce an electrical current for direct delivery to the stimulation electrodes 345a to 345n without the use of a signal processor. This embodiment also encompasses future generations of circuitry that may provide for a sound processing circuitry to be carried “on-chip” with the sensor.

Those skilled in the art will appreciate that the exemplary systems, combinations and descriptions are merely illustrative of the invention as a whole, and that variations of components, dimensions, and compositions described above may be made within the spirit and scope of the invention. Specific characteristics and features of the invention and its method are described in relation to some figures and not in others, and this is for convenience only. While the principles of the invention have been made clear in the exemplary descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

1. An implant body for coupling to middle or inner ear structure, the body carrying a wafer scale inertial sensor having a piezoresistive-doped cantilever coupled to a seismic mass for detecting acoustic waves in the ear structure.

2. An implant body as in claim 1 further comprising signal circuitry coupling the piezoresistive-doped cantilever to a signal processor.

3. An implant body as in claim 2 further comprising a cochlear implant portion coupled by circuitry to the signal processor.

4. An ossicular implant comprising an inertial sensor chip that defines a flexure coupled to a suspended mass, the flexure carrying a piezoresistive element coupled to signal circuitry that extends to an off-chip signal processor for sensing acoustic waves in middle ear structure.

5. The ossicular implant as in claim 4 further comprising a cochlear implant coupled by circuitry to the signal processor.

6. A sensor for implantation in ear structure comprising at least one wafer scale deflectable flexure portion coupled to a suspended mass portion wherein the flexure carries a piezoelectric element.

7. The sensor as in claim 6 further comprising signal circuitry coupling the piezoelectric element to a signal processor.

8. The sensor as in claim 7 further comprising a cochlear implant coupled by circuitry to the signal processor.

9. An implant for treating hearing disorders comprising an implant body of a biocompatible material for coupling to hearing structure between and including the eardrum and the cochlea, and a micro-fabricated sensor system within the implant body comprising a deflectable cantilever coupled to a suspended mass, a portion of the cantilever doped with a piezoelectric or piezoresistive material.

10. A method for treating a hearing disorder of a human patient, comprising the steps of;
(a) providing an implant body that carries at least one wafer scale inertial sensor having a piezoresistive-doped flexure coupled to a suspended mass; and
(b) acquiring input signals of acoustic pressure waves within middle or inner ear structure by detecting changes in resistance to current flow through each piezoresistive-doped flexure during deflection of the flexure and suspended mass in response to acoustic displacements.

11. A method as in claim 10 wherein step (b) acquires input signals associated with acoustic displacements in a single axis.

12. A method as in claim 10 wherein step (b) acquires input signals associated with acoustic displacements in two axes.

13. A method as in claim 10 wherein step (b) acquires input signals associated with acoustic displacements in three axes.

14. A method as in claim 10 further comprising the step of processing the input signals with a signal processor.

15. A method as in claim 11 further comprising the step of filtering the input signals.

16. A method as in claim 11 further comprising the step of amplifying the input signals.

17. A method as in claim 11 further comprising the step of digitizing the input signals.

18. A method as in claim 11 further comprising the step of utilizing the signal processor to provide coded output signals for delivery to a cochlear implant.

19. A method as in claim 11 further comprising the step of utilizing the signal processor to provide output signals to deliver electrical energy to an electrode array carried by a cochlear implant to stimulate auditory nerve fibers in the cochlea.

20. A method as in claim 11 further comprising the step of utilizing the signal processor to provide output signals to deliver electrical energy to an electrode array carried by a cochlear implant to stimulate auditory nerve fibers in the cochlea.

21. A method for treating a hearing disorder of a human patient, comprising the steps of;
(a) coupling an implant body to middle ear structure that carries a wafer scale inertial sensor with a flexure coupled to a suspended mass, the flexure carrying a piezoelectric element;
(b) permitting acoustic waves to deflect the flexure and suspended mass; and
(c) detecting electrical current flow from the piezoelectric element to thereby provide input signals of the acoustic pressure waves.

22. A method as in claim 21 wherein step (c) detects pressure waves along a single axis.

23. A method as in claim 21 wherein step (c) detects pressure waves along multiple axes.

24. A method as in claim 21 further comprising the step of processing the input signals with a signal processor and transmitting output signals to a cochlear implant.
